(19) United States
(12) Patent Application Publication
(10) Pub. No.: US 2023/0201272 A1
(43) Pub. Date: Jun. 29, 2023

(54) CELL-BASED THERAPIES AND USES THEREOF

(71) Applicant: Tandem Biotherapeutics, Inc., Wayland, MA (US)

(72) Inventors: Roberto Gramignoli, Stockholm (SE); Stephen Strom, Stockholm (SE)

(73) Assignee: Tandem Biotherapeutics, Inc., Wayland, MA (US)

(21) Appl. No.: 17/923,652

(22) PCT Filed: May 6, 2021
(86) PCT No.: PCT/US21/31123

§ 371 Date: Nov. 7, 2022

Related U.S. Application Data
(60) Provisional application No. 63/021,929, filed on May 8, 2020.

(51) Int. Cl. A61K 35/50; C07K 14/705; A61K 9/00

(52) U.S. Cl. CPC A61K 35/50; C07K 14/70585; C07K 14/70589; C07K 14/7055; A61K 9/0019

(57) ABSTRACT

Aspects of the disclosure relate to compositions comprising populations of isolated amniotic cells. In some embodiments, the populations of cells are enriched for human amniotic epithelial cells (hAECs). In some embodiments, the disclosure provides methods of administering the compositions to a subject, for example a subject having certain diseases or disorders such as liver disease, phenylketonuria (PKU), a vocal cord injury or a disease associated with a Complement Factor H deficiency.

### CROSS-REFERENCE TO RELATED APPLICATIONS

This application is a national stage filing under 35 U.S.C. § 371 of international PCT application PCT/US2021/031123, filed May 6, 2021, which claims priority under 35 U.S.C. § 119(e) to U.S. provisional patent application, U.S. Ser. No. 63/021,929, filed May 8, 2020, the entire contents of each of which are incorporated by reference herein.

### TECHNICAL FIELD

The technology described herein relates to cell therapy.

### BACKGROUND

Cellular therapeutics have potential utility for a large number of unmet medical needs. Large-scale, clinical-grade isolation and subsequent banking of stem cells has been the backbone of the field of regenerative medicine. Successful translation of cell-based therapies frequently requires successful long-term storage under conditions that maintain the safety and efficacy of the final cell product. Cell cryopreservation is an established procedure for several clinical therapies, as decades of bone marrow and cord blood hematopoietic stem cell therapies have shown. Nevertheless, epithelial cell-based treatments, including hepatocytes, have suffered from limited reanimation and function and with considerable loss of viability.

### SUMMARY

Described herein are compositions comprising populations of cells enriched for human amniotic epithelial cells (hAECs). Also described are methods of producing compositions comprising populations of cells characterized by a high percentage of hAECs relative to other cell types (e.g., mesenchymal stromal/stem cells (MSCs) or endothelial cells).

In some aspects, the disclosure provides a composition comprising (1) a population of isolated cells comprising (that is) at least 70% human amniotic epithelial cells (hAECs), wherein no more than 2% of the cell population is hematopoietic cells and less than 1% of the remaining cells are negative for HLA-DR, and (2) a pharmaceutically acceptable excipient. In some embodiments, the population of isolated cells comprises 80%, 90% or 95% hAECs, wherein no more than 2% of the cell population is hematopoietic cells and less than 1% of the remaining cells are negative for HLA-DR.

In some aspects, the disclosure provides a composition comprising (1) a population of isolated cells comprising (that is) at least 95% human amniotic epithelial cells (hAECs) and (2) a pharmaceutically acceptable excipient.

In some embodiments, the isolated cells are capable of adhering to proteins of the extracellular matrix (ECM). Examples of ECM proteins include, but are not limited to, laminin or collagen isoforms, fibronectin, elastin, gelatin, hyaluronic acid or hyaluronic acid-binding proteins, and/or Engelbreth-Holm-Swarm (EHS) Matrix Extract.

In some embodiments, less than 30% of the cells in the population express a stromal cell marker. In some embodiments, less than 2% of the cells in the population express a stromal cell marker. In some embodiments, the stromal cell marker is CD44 (HCAM) or CD105 (endoglin). In some embodiments, less than 30% of the cells in the population express CD44 and CD105. In some embodiments, less than 2% of the cells in the population express CD44 and CD105.

In some embodiments, less than 2% of the cells in the population express a hematopoietic cell marker. In some embodiments, less than 1% of the cells in the population express a hematopoietic cell marker. In some embodiments, the hematopoietic cell marker is CD45.

In some embodiments, the hAECs express one or more cell surface markers selected from CD326 (EpCAM), CD29 (betal-integrin), CD106 (VCAM-1), MadCAM-1, CD51 (alphaV integrin), CD166 (ALCAM), CD324 (E-cadherin), CD49f (alpha6 integrin), CD104 (beta4 integrin), CD73, CD47, CD59 (DAB), CD55 (DAF), CD81, CD9, CD29L, SSEA-4, CD109, CD298 (ATP1B3), and Na/K transporting ATPase subunit beta3. In some embodiments, the one or more cell surface markers is CD326 or CD49f.

In some embodiments, the hAECs express one or more HLA-Ia molecules. In some embodiments, the HLA-Ia molecule is HLA-A. In some embodiments, the hAECs express (a) HLA-G, (b) HLA-E, or (c) HLA-G and HLA-E. In some embodiments, the HLA-G is soluble HLA-G, the HLA-E is soluble HLA-E, or the HLA-G is soluble HLA-G and the HLA-E is soluble HLA-E. In some embodiments, the hAECs do not express any HLA class II antigens.

In some embodiments, the population of cells (e.g., population of isolated cells comprising at least 70% human amniotic epithelial cells (hAECs)) has previously been cryopreserved and subsequently thawed. In some embodiments, at least 80% of the hAECs are viable after having been cryopreserved and subsequently thawed. In some embodiments, at least 90%, 95%, or 99% of the hAECs are viable after having been cryopreserved and subsequently thawed.

In some embodiments, the hAECs are present at a density between about 1×10⁶ cells/ml and about 1×10⁸ cells/ml. In some embodiments, the hAECs are present at a density between about 1×10⁶ cells/ml and about 1×10⁷ cells/ml.

In some embodiments, the pharmaceutically acceptable excipient comprises one or more of the following: physiological saline, heparin, and human serum albumin (HSA). In some embodiments, the heparin is present in an amount of about 10 U/ml. In some embodiments, the HSA is present in the composition in an amount of about 0.5% v/v. In some embodiments, the composition comprises less than 0.1% DMSO.

In some aspects, the disclosure provides a method for producing a cell preparation comprising an enriched population of human amniotic epithelial cells (hAECs), the method comprising: combining amniotic membrane tissue with an enzyme that digests amniotic membrane tissue under conditions, such as shaking (e.g., rotating) conditions, that result in release of a population of cells; suspending (combining) the population of cells in (with) a first buffer comprising albumin, thereby producing a first buffer (optionally comprising albumin) and a population of cells; removing the first buffer (and optionally albumin) and resuspending the cells in a saline solution to produce a cell preparation; filtering the cell preparation produced (e.g., through a 100 μm filter) into one or more containers comprising a DMSO-free cryopreservation (cryogenic) solution, thereby producing a cell preparation comprising an enriched population of human amniotic epithelial cells (hAECs). In some embodiments, the method further comprises placing the one or more containers in a temperature-controlled storage apparatus, wherein the temperature of the apparatus is reduced at a rate of 1° C. per minute until a final temperature of lower than −80° C. is reached. In some embodiments, a final temperature of between −80° C. and −237° C. is reached. In some embodiments, a final temperature of between −80° C. and −210° C. is reached.

In some aspects, the disclosure provides a method for producing a cell preparation comprising an enriched population of human amniotic epithelial cells (hAECs), the method comprising: combining amniotic membrane tissue with an enzyme that digests amniotic membrane tissue under conditions, such as shaking (e.g., rotating) conditions, that result in release of a population of cells; suspending (combining) the population of cells produced in (with) a first buffer comprising albumin, thereby producing a combination of first buffer (optionally comprising albumin) and a released population of cells; removing the first buffer (and optionally albumin) from the combination and resuspending the cells obtained in a saline solution, producing a cell preparation; filtering the cell preparation produced (e.g., through a 100 μm filter) into one or more containers (e.g., vial) along with a cryopreservation solution/media, such as a DMSO-free cryopreservation solution, thereby producing a cell preparation comprising an enriched population of human amniotic epithelial cells (hAECs) in cryopreservation (cryogenic) solution. In some embodiments, the method further comprises cryopreserving the cell preparation of enriched population of hAECs in cryopreservation solution: the one or more containers containing the cell preparation in cryopreservation solution are placed in a temperature-controlled apparatus, such as a storage apparatus, and the temperature of the apparatus is reduced, such as at a rate of 1° C. per minute, until a final temperature of between 0° C. and −237° C. is reached. In some embodiments, a final temperature of between −60° C. and −237° C. is reached. In some embodiments, a final temperature of between −60° C. and −210° C. is reached. In some embodiments, a final temperature of between −60° C. and −90° C. is reached. In some embodiments, a final temperature of between 0° C. and −90° C. is reached. Once frozen, the containers (vials) containing cell preparation in cryopreservation solution can be preserved at any temperature below about −70° C. Classical preservation in liquid nitrogen (−196° C.) or in vapor phase (−156° C.) is not necessary; cell preparations can be preserved, for example, at −80° C.

In some embodiments, the final temperature is maintained for a minimum of 30 days. In some embodiments, the final temperature is maintained up to 18 years.

In some embodiments, the amniotic membrane tissue is combined with a proteolytic enzyme. In some embodiments, the digesting comprises incubating the amniotic membrane tissue with trypsin. In some embodiments, the digesting comprises incubating the amniotic membrane tissue with a protease, or a protease mixture. In some embodiments, the amniotic membrane tissue is combined with a protease that cleaves integrin. Examples of proteolytic enzymes include, but are not limited to trypsin, TrypLE™, dispase, Accutase™, DNAse solutions, calpain or a calpain-like protease, or any combination or mixture thereof. In some embodiments, the proteolytic enzyme or the protease mixture is supplemented with a chelating agent. In some embodiments, the proteolytic enzyme is supplemented with a chelator of divalent cations. In some embodiments, the proteolytic enzyme is supplemented with a chelator of calcium. In some specific embodiments, the proteolytic enzyme is supplemented with EGTA or EDTA. In some embodiments, the protease or protease mixture further comprises a chelating agent, optionally wherein the chelating agent comprises or consists of EGTA or EDTA.

In some embodiments, the method further comprises the step of thawing the cell preparation to between 4° C. and 37° C. In some embodiments, the method further comprises the step of thawing the cell preparation to between 4° C. and room temperature. In some embodiments, the method further comprises the step of removing the cryopreservation solution (e.g., cryoprotectant) and suspending (or resuspending) the cell preparation in a pharmaceutically acceptable excipient to produce a pharmaceutical composition. In some embodiments, the pharmaceutical composition comprises at least 70% human amniotic epithelial cells (hAECs). In some embodiments, the pharmaceutical composition comprises at least 90% human amniotic epithelial cells (hAECs). In some embodiments, the pharmaceutical composition comprises at least 95% human amniotic epithelial cells (hAECs). In some embodiments, the pharmaceutical composition comprises at least 70% human amniotic epithelial cells (hAECs), wherein no more than 2% of the cell population is hematopoietic cells, and less than 1% of the remaining cells are negative for HLA-DR. In some embodiments, the pharmaceutical composition comprises at least 1×10⁶ hAECs. In some embodiments, the pharmaceutical composition comprises at least 5×10⁶ hAECs. In some embodiments, the pharmaceutical composition comprises at least 1×10⁸ hAECs.

In some aspects, the disclosure provides a pharmaceutical composition made by a method described herein.

In some aspects, the disclosure provides a method for treating a liver-associated disease, a vocal cord injury or a disease associated with a Complement Factor H deficiency in a subject in need thereof, the method comprising administering to the subject in need thereof a composition such as the isolated cell compositions described herein, including a cell composition that was cryopreserved and subsequently thawed.

In some embodiments, the liver-associated disease is acute liver disease, chronic liver disease, or an inborn error of metabolism.

In some embodiments, the subject is a mammal. In some embodiments, the subject is a human.

In some embodiments, the administration is via direct injection to the liver of the subject. In some embodiments, the direct injection is portal vein injection, hepatic artery injection, or mesenteric vein injection.

In some aspects, the disclosure provides a method for treating an inborn error of metabolism (such as phenylketonuria (PKU)) in a subject in need thereof, the method comprising administering to the subject having the inborn error of metabolism (e.g., PKU) a composition as described herein. In some aspects, the disclosure provides a method for treating phenylketonuria (PKU) in a subject in need thereof, the method comprising administering to the subject having PKU a composition as described herein.

In some embodiments, the administration results in expression of an enzyme of interest, (e.g., phenylalanine hydroxylase (PAH) enzyme) in the liver of the subject. In some embodiments, the administration results in a reduction in a level of a substrate of the enzyme of interest (e.g., phenylalanine (PHE)) in the blood of the subject relative to the level in the subject's blood prior to the administration. In some embodiments, the administration results in expression of phenylalanine hydroxylase (PAH) enzyme in the liver of the subject. In some embodiments, the administration results in a reduction of phenylalanine (PHE) in the blood of the subject relative to the PHE level in the subject's blood prior to the administration.

In some embodiments, the subject is not in a state of immunosuppression prior to the administration. In some embodiments, the subject is a human. In some embodiments, a subject is in a state of immunosuppression (e.g., has been administered one or more immunosuppressive agents) prior to the administration.

In some embodiments, the administration is via direct injection to the liver of the subject. In some embodiments, the direct injection is portal vein injection or mesenteric vein injection.

### BRIEF DESCRIPTION OF DRAWINGS

FIG. 1 shows cell viability and recovery after cryopreservation. Cell viability of hAECs isolated from 14 different full-term human placentae are represented as grey bars. The average viability of fresh hAEC is 90%±4% and it is also shown (AVG). Similarly, hAEC viability after cryopreservation is represented as black bars, with an average value of 78%±5%. Cell recovery for each case is shown as the black and white, checkered bar and is presented as a percentage of the total number of cells initially cryopreserved (10 million, right axis) with average value of 0.65±0.11×10⁷/ml. * p<0.001, t-test on paired values.

FIGS. 2A-2D show fluorescence-assisted cell sorting (FACS) analysis on fresh and cryopreserved hAECs. FIG. 2A shows a representative gating strategy, where forward and side scatters identified intact cells, an additional gate selects single cells, and is specifically gated for different surface markers using two different fluorochromes. Every case was characterized by specific immune-labelling with primary antibodies directed against CD31; CD44; CD45; CD49f; CD105; CD326. The negative isotype control for the respective fluorochromes is shown in the small dot plot graph for each marker. FIG. 2B shows cells positive for CD49f (integrin α6 subunit) 99%±1% or CD326 (EpCAM) 88%±8% and 91%±6%, respectively) both before and after cryopreservation. FIG. 2C shows amnion-derived MSC were identified based on their expression of characteristic markers (CD44 and CD105). FIG. 2D shows the hematopoietic marker CD45 was measured on 1.2%±1.4% and 0.3%±0.4% of the cells in suspensions obtain after isolation and after cryopreservation, respectively. All values measured in 14 preparations are reported, with an average±SD as last bar on the right. * p<0.001, t-test on paired values.

FIGS. 3A-3B show data relating to immunomodulatory molecule and ectoenzyme expression on cryopreserved cells. FIG. 3A shows HLA-ABC was expressed on average of 39%±18% in all cells; HLA-DR is undetectable in all cases; HLA-G was expressed on all cases of cryopreserved hAEC with an average of 79%±9% of the cells. Ectoenzymes CD39 and CD73 were expressed on average on 30%±7% and 88%±7% of the cells in all cases, respectively. FIG. 3B shows the ectoenzymes CD38, CD39 and CD73 were expressed in all cryopreserved hAEC cases.

FIGS. 4A-4B show stem cell transcription factors and extracellular matrix (ECM) protease expression on cryopreserved cells. FIG. 4A shows the degree of plasticity in hAEC is estimated with stem cell markers commonly measured in embryonic stem cells: SOX2, OCT4, NANOG and DLK-1. All four genes were expressed in all hAEC cases. FIG. 4B shows the matrix metalloprotease (MMP) -2, -3, and -9 were expressed in all hAEC cases, whereas MMP-7, MMP-8 and MMP-13 were undetectable. One preparation expressed MMP-12. The tissue inhibitor of metalloproteinases (TIMP)-1 is highly expressed in all cryopreserved hAEC.

### DETAILED DESCRIPTION

Aspects of the disclosure relate to compositions comprising populations of cells enriched for human amniotic epithelial cells (hAECs). The disclosure is based, in part, on methods of producing compositions comprising populations of cells characterized by a high percentage of hAECs relative to other cell types, for example mesenchymal stromal/stem cells (MSCs) or endothelial cells. In some embodiments, compositions described by the disclosure (e.g., compositions comprising enriched populations of hAECs) are useful for treating certain diseases and disorders, for example liver diseases, PKU, a vocal cord injury or a disease associated with a Complement Factor H deficiency.

### Preparation of hAECs

Aspects of the disclosure relate to methods for isolating populations of cells. In some embodiments, methods described herein produce populations of cells (and compositions comprising populations of cells) that are enriched in a particular cell type. As used herein, “enrichment” or a population of cells that is “enriched” refers to a population of cells comprising an increased amount, for example by percentage of a population, of one specific cell type relative to the amount of other cell types in the population. An “enriched” population of cells may comprise at least 20%, 30%, 40%, 50%, 60%, 70%, 80%, 90%, 95%, 98%, 99%, or 100% of a desired cell type relative to the other types of cells in the population. In some aspects, the disclosure relates to methods of producing populations of cells enriched in human amniotic epithelial cells (hAECs).

Accordingly, in some aspects, the disclosure provides a method for producing a cell preparation comprising an enriched population of human amniotic epithelial cells (hAECs), the method comprising: combining amniotic membrane tissue with an enzyme that digests amniotic membrane tissue under conditions, such as shaking (e.g., rotating) conditions, that result in release of a population of cells; suspending the cells in a first buffer (optionally wherein the first buffer comprises albumin); removing the first buffer (and optionally albumin) and resuspending the cells in a saline solution to produce a cell preparation comprising hAECs; filtering the cell preparation (e.g., through a 100 μm filter) into one or more containers comprising a DMSO-free cryopreservation solution, thereby producing a cell preparation comprising an enriched population of human amniotic epithelial cells (hAECs). In some embodiments, the method further comprises placing the one or more containers in a temperature-controlled storage apparatus, wherein the temperature of the apparatus is reduced at a rate of 1° C. per minute until a final temperature of lower than −60° C. is reached.

As described further in the Examples, methods described herein (and the enriched cell populations produced by such methods) have several advantages over previously utilized methods for producing populations of amniotic cells, for example increased cost- and time-efficiency and increased purity. In some embodiments, methods described by the disclosure comprise a single hAEC isolation step, whereas previously utilized methods require two or three separate isolation steps. In some embodiments, the proteolytic enzyme is used in combination with a chelating agent, such as EGTA or EDTA, in the amniotic membrane digestion step. It was observed that when the method was carried out using a proteolytic enzyme and without EGTA or EDTA, few cells were released. It is hypothesized that the presence of a chelating agent, such as EGTA or EDTA, facilitates the release of cells from the amniotic membrane by disrupting cell-to-cell adhesion. In some embodiments, the amount of proteolytic enzyme used in a digestion is adjusted (e.g., the concentration is increased or decreased) to compensate for any effect that the chelating agent has on its activity, in order to enhance or increase cell release.

Additionally, in some embodiments, methods described herein comprise a single, 30-minute enzymatic incubation step. In some embodiments, methods described by the disclosure produce populations of cells comprising at least 70% hAECs. In some aspects, the disclosure provides a composition comprising (1) a population of isolated cells comprising (that is) at least 70% human amniotic epithelial cells (hAECs), wherein no more than 2% of the cell population is hematopoietic cells and less than 1% of the remaining cells are negative for HLA-DR, and (2) a pharmaceutically acceptable excipient. In some embodiments, the population of isolated cells comprises 80%, 90% or 95% hAECs, wherein no more than 2% of the cell population is hematopoietic cells and less than 1% of the remaining cells are negative for HLA-DR.

In some aspects, the disclosure provides a composition comprising (1) a population of isolated cells comprising (that is) at least 95% human amniotic epithelial cells (hAECs) and (2) a pharmaceutically acceptable excipient.

In some embodiments, the isolated cells are capable of adhering to proteins of the extracellular matrix (ECM), such as: laminin or collagen isoforms, fibronectin, elastin, gelatin, hyaluronic acid or hyaluronic acid-binding proteins, and/or EHS Matrix Extract.

Amniotic cells (e.g., hAECs) are isolated from a placenta obtained from a subject. In some embodiments, the subject is a human. In some embodiments, obtaining amniotic cells comprises extracting (e.g., isolating or physically separating) amniotic cells from the amniotic membrane of a placenta. In some embodiments, obtaining comprises washing an amniotic membrane (e.g., an amniotic membrane separated from the rest of the placenta) with an isotonic solution. In some embodiments, the isotonic solution is Ringer solution (or an equivalent thereof). In some embodiments, the Ringer solution comprises sodium chloride, potassium chloride, calcium chloride, and sodium bicarbonate. In some embodiments, the Ringer solution is between pH 5 and pH 8 (e.g., about pH 5, about pH 6, about pH 7, about pH 7.5, about pH 8, etc.). In some embodiments, the Ringer solution further comprises a chelating agent. In some embodiments, the chelating agent is ethylene glycol-bis(β-aminoethyl ether)-N,N,N′,N′-tetraacetic acid (EGTA). In some embodiments, the chelating agent is present in the Ringer solution at a concentration between about 2 mM and about 6 mM (e.g., about 2 mM, 3 mM, 4 mM, 5 mM, 6 mM, etc.). In some embodiments, the amniotic membrane is washed with Ringer solution 1, 2, 3, 4, 5, 6, 7, 8, 9, 10, or more times. In some embodiments, the amniotic membrane is washed until no blood or blood clots remain associated with the amniotic membrane. In some embodiments, the amniotic membrane is cut into pieces weighing approximately 1.5-2 gr, and optionally suspended in 2-3 ml volume saline.

The amniotic membrane (e.g., pieces of the amniotic membrane) are subjected to enzymatic digestion. In some embodiments, the digesting comprises contacting the amniotic membrane with a proteolytic enzyme (also referred to as a protease). Examples of proteolytic enzymes include serine proteases (e.g., trypsin, chymotrypsin, thrombin, elastase, subtilisin, etc.), cysteine proteases (e.g., calpain, papain, cathepsin k, caspase-1, separate, TEV protease, etc.), threonine proteases (e.g., Ntn fold proteasomes, ornithine acyltransferases, etc.), aspartic proteases (e.g., pepsin, signal peptidase II, presinilin, GPR endopeptidase, omptin, etc.), glutamic proteases (e.g., G1, G2, etc.), metalloproteases (e.g., ADAM, MMP-2, MMP-9, MMP-11, MMP-19), collagenases, dispase, Accutase™, hyaluronidase, DNAse solution, calpain or calpain like proteases, etc. In some embodiments, the proteolytic enzyme is trypsin. In some embodiments, the proteolytic enzyme is a recombinant enzyme. In some embodiments, the trypsin is recombinant trypsin, for example TrypLE enzyme.

The amount, concentration, or activity of protease used to digest the amniotic membrane can vary. In some embodiments, enzymatic activity is equal to moles of substrate converted per unit time (e.g., enzyme unit, 1 μmol min⁻¹). In some embodiments, the amniotic membrane is digested with an enzyme having an activity ranging from about 1× enzyme to about 10× enzyme (e.g., 1×, 2×, 3×, 4×, 5×, 6×, 7×, 8×, 9×, or 10× enzyme). In some embodiments, the enzyme used to digest the amniotic membrane is 10× TrypLE enzyme.

In some embodiments, the proteolytic enzyme is supplemented with a chelating agent. In some embodiments, the proteolytic enzyme is supplemented with a chelator of divalent cations.

In some embodiments, the proteolytic enzyme is supplemented with a chelator of calcium. In some specific embodiments, the proteolytic enzyme is supplemented with EGTA or EDTA. Examples of chelating agents include, but are not limited to: EGTA (ethylene glycol-bis(β-aminoethyl ether)-N,N,N′,N′-tetraacetic acid), EDTA (Ethylenediaminetetraacetic acid), 1,10-phenanthroline, DTPA (diethylenetriaminepentaacetic acid), BAPTA (1,2-bis(o-aminophenoxy)ethane-N,N,N′,N′-tetraacetic acid). Without wishing to be bound by theory, the use of a chelating agent such as EGTA or EDTA is typically contra-indicated when proteases are used because these chelators deplete calcium ions, which are required for the functioning of several enzymes. Typically, extracellular matrix digestion and cell release is achieved using enzymatic solutions. The isolation method disclosed herein is different from standard techniques at least because the proteolytic enzyme is used in combination with a chelating agent, such as EGTA or EDTA, in the amniotic membrane digestion step. It was observed that when the method was carried out using a proteolytic enzyme and without EGTA or EDTA, few cells were released. It is hypothesized that the presence of a chelating agent, such as EGTA or EDTA, facilitates the release of cells from the amniotic membrane by disrupting cell-to-cell adhesion. In some embodiments, the amount of proteolytic enzyme used in a digestion is adjusted (e.g., the concentration is increased or decreased) to compensate for any effect that the chelating agent has on its activity, in order to enhance or increase cell release.

The length of incubation with the proteolytic enzyme can vary. In some embodiments, an amniotic membrane is incubated with a protease for between about 15 minutes and about 60 minutes (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes). In some embodiments, an amniotic membrane is incubated with a protease for more than 60 minutes (e.g., 75, 90, 120 minutes, etc.).

The disclosure is based, in part, on the recognition that digesting amniotic membrane tissue with a protease under shaking conditions improves the amount of hAECs present in the final isolated cell population. Without wishing to be bound by any particular theory, the combination of certain enzymes (e.g., trypsin, TrypLE, etc.) with shaking conditions results in the release of superficial epithelial cells (e.g., hAECs) without dissolving membrane architecture. This is advantageous because it reduces the release of other cell types (e.g., mesenchymal stem cells) embedded in the amnion stroma. In some embodiments, the shaking conditions comprise shaking every 10 minutes during the digestion. In some embodiments, the digestion reaction (e.g., an amniotic membrane being contacted with a protease) is shaken 1, 2, 3, 4, 5, or more times. The duration of the shaking can vary. In some embodiments, the shaking lasts for between about 10 seconds and about 60 minutes. In some embodiments, the shaking comprises continuous agitation (e.g., rotation) for 30 min. In some embodiments, the continuous agitation may range from about 1 rpm to about 50 rpm. In some embodiments, the continuous agitation (e.g., rotation) is performed at a velocity of 35 rotations per minute (rpm). In some embodiments, after shaking, the amniotic membrane is agitated (e.g., using forceps) to release remaining cells (e.g., hAECs) from the membrane.

In some embodiments, the cells are suspended in a buffer after the digesting step. In some embodiments, buffer solution is at 4° C. In some embodiments, the buffer comprises albumin. The disclosure is based, in part, on the recognition that albumin supplementation contributes to inactivating remaining enzyme (e.g., protease) in the cell suspension during cell washes. The concentration of albumin in the buffer can vary. In some embodiments, the concentration of albumin ranges from about 0% (e.g., not present) to about 2% v/v. In some embodiments, the cells are centrifuged to remove the buffer (e.g., buffer containing albumin) and resuspended in a saline solution. In some embodiments, the albumin is completely removed. In some embodiments, cells comprise a residual amount of albumin (e.g., less than 1%, less than 0.5%, less than 0.1% albumin, etc.). In some embodiments, the solution is resuspended by swirling the cells in the saline solution. In some embodiments, the resuspending does not comprise shaking the cells, since vigorous shaking may negatively affect cell viability.

In some embodiments, resuspended cells (also referred to as a cell preparation) are filtered (e.g., mechanically separated from other membrane components). In some embodiments, the filtering comprises passing the cell preparation through one or more (e.g., 1, 2, 3, 4, 5, or more) filters into one or more containers. The size of the filters can vary. In some embodiments, the size of the filter ranges from about 10 μm to about 200 μm (e.g., any filter size between 10 μm and 200 μm, inclusive. In some embodiments, one or more 100 μm filters are used.

In some embodiments, the one or more containers comprise a cryopreservation solution (e.g., a solution comprising a cryoprotectant). Examples of cryoprotectants include glycols (e.g., ethylene glycol, propylene glycol, glycerol, etc.), dimethyl sulfoxide (DMSO), certain sugars (e.g., trehalose, etc.), and combinations thereof. In some embodiments, a cryopreservation solution comprises DMSO. In some embodiments, a cryopreservation solution lacks DMSO (e.g., is DMSO-free). In some embodiments, the concentration of DMSO in a composition (e.g., a cell preparation) is less than about 10% v/v (e.g., 9%, 8%, 7%, 6%, 5%, 4%, 3%, 2%, 1%, or less than 1%). In some embodiments, the concentration of DMSO in a composition (e.g., a cell preparation) is less than about 0.1% v/v. In some embodiments, a cryoprotectant lacks (e.g., does not comprise) DMSO.

In some embodiments, a composition comprising a population of cells (e.g., a population of cells comprising hAECs) and one or more cryoprotectants is placed in a temperature-controlled storage apparatus (e.g., a freezer, a liquid nitrogen storage tank, etc.) for freezing. In some embodiments, the temperature of the apparatus is reduced at a rate of 1° C. per minute until a final temperature of lower than −60° C. (e.g., −70° C., −80° C., −100° C., −156° C., etc.) is reached. In some embodiments, the temperature ranges from about −60° C. to about −210° C. In some embodiments, the final temperature is maintained for a minimum of 30 days (e.g., 45 days, 60 days, 120 days, 365 days, etc.). In some embodiments, the temperature is maintained for between 1 year and 18 years.

The disclosure is based, in part, on the recognition that thawing cryopreserved cell populations (e.g., cryopreserved cell populations comprising hAECs) results in the enrichment of hAECs within the cell population. In some embodiments, the cell preparation is thawed to between 4° C. and 37° C. In some embodiments, the cryoprotectant is removed after thawing and the cells are resuspended in a pharmaceutically acceptable excipient to produce a pharmaceutical composition (e.g., a pharmaceutical composition comprising an enriched population of hAECs). In some embodiments, isolation methods described herein result in a yield of hAECs between about 5×10⁶ cells/gram and 5×10⁷ cells/gram amniotic tissue.

### Human Amniotic Epithelial Cells (hAECs)

The disclosure relates, in part, to compositions of isolated cells. As used herein, “isolated” refers to a cell, or a collection of the same type of cells, that has been removed from tissue and separated (e.g., physically separated) from cells of other types. Isolation of cells typically results in an enrichment (e.g., an increased amount, for example by percentage of a population) of the isolated cell type relative to the amount of other cell types in a population. In some embodiments, a collection of isolated cells (also referred to as a population of isolated cells) comprises an amount or concentration of isolated cells that is higher than could be found naturally in any tissue or organism. In some embodiments, an isolated cell (or population of isolated cells) is enriched for the presence of human amniotic epithelial cells (hAECs).

As used herein, the term “human amniotic epithelial cells” or “hAECs” (also called amnion epithelial cells or multipotent epithelial cells) refers to a multipotent cell type obtained (e.g., isolated) from the amniotic membrane of a human placenta. hAECs are not obtained from amniotic fluid, umbilical cord tissue, or other types of placental tissue.

In some embodiments, hAECs are identified (or characterized) by the presence of certain cell surface markers, also referred to as cell surface antigens. Cell surface markers are molecules (e.g., peptides, proteins, polypeptides, carbohydrates, etc.) that are expressed on the surface of the cell and, in some embodiments, are cell-type specific (e.g., serve as an identifying characteristic of a particular cell type for immunophenotyping). In some embodiments, a cell surface marker is anchored (e.g., covalently connected) to the membrane of the cell by which it is expressed. In some embodiments, a cell surface protein is a transmembrane protein, cell surface receptor, or a cell surface receptor ligand. Examples of cell surface receptors include CD3, CD4, CD8, CD19, CD20, CD123, CD56, CD34, CD14, CD33, CD66b, CD41, CD61, CD146, CD326 (EpCAM), CD29 (betal-integrin), VCAM-1, MadCAM-1, CD51 (alpha V integrin), CD166 (ALCAM), CD324 (E-cadherin), CD325 (N-cadherin), CD49f (alpha6 integrin), CD104 (beta4 integrin), CD73, CD47, CD59 (DAB), CD55 (DAF), CD81, CD9, CD29L, SSEA-4, CD109, CD105 (endoglin), CD298 (ATP1B3), CD90, CD31 (PECAM), CD45, CD235 (glycophorin), and Na/K transporting ATPase subunit beta3. In some embodiments, the isolated hAEC cells are capable of adhering to proteins of the extracellular matrix. Examples of extracellular matrix proteins include but are not limited to: laminin or collagen isoforms, fibronectin, elastin, gelatin, hyaluronic acid or hyaluronic acid-binding proteins, and/or EHS Matrix Extract. EHS Matrix Extract is a solubilized basement membrane preparation extracted from the Engelbreth-Holm-Swarm (EHS) mouse sarcoma.

The disclosure is based, in part, on populations of cells that comprise greatly reduced numbers (or completely lack) cells comprising cell surface markers that are present on stromal cells. Examples of stromal cells include connective tissue cells, for example mucosal cells, bone marrow cells, lymph node cells, mesenchymal stem cells (MSCs), etc. The number of cells comprising stromal cell markers in a cell population may vary. In some embodiments, less than 30%, less than 25%, less than 20%, less than 25%, less than 10%, less than 9%, less than 8%, less than 7%, less than 6%, less than 5%, less than 4%, less than 3%, less than 2%, or less than 1% of cells in a population comprise a stromal cell marker. In some embodiments, less than 2% (e.g., 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, or 0.1%) of the cells in the population express a stromal cell marker. In some embodiments, none of the cells in a population comprise a stromal cell marker (e.g., the population of cells lacks stromal cells). In some embodiments, the stromal cell marker is CD44 or CD105.

The disclosure is based, in part, on populations of cells that comprise less than 2% of cells comprising cell surface markers that are present on hematopoietic cells (e.g., erythroid cells, myeloid cells, hematopoietic stem cells, etc.). Examples of hematopoietic cells include erythrocytes (mature red blood cells, reticulocytes, etc.), lymphocytes (white blood cells, T-cells, B-cells, natural killer (NK) cells, etc.), myeloid cells (e.g., granulocytes, megakaryocytes, macrophages, etc.), hematopoietic stem cells (HSCs), etc. The number of cells comprising hematopoietic cell markers in a cell population may vary. In some embodiments, less than 10%, less than 9%, less than 8%, less than 7%, less than 6%, less than 5%, less than 4%, less than 3%, less than 2%, or less than 1% of cells in a population comprise a hematopoietic cell marker. In some embodiments, less than 1% (e.g., 0.9%, 0.8%, 0.7%, 0.6%, 0.5%, 0.4%, 0.3%, 0.2%, or 0.1%) of the cells in the population express a hematopoietic cell marker. In some embodiments, none of the cells in a population comprise a hematopoietic cell marker (e.g., the population of cells lacks hematopoietic cells). In some embodiments, the hematopoietic cell marker is CD45.

In some embodiments, the hAECs express one or more cell surface markers selected from CD326 (EpCAM), CD29 (betal-integrin), VCAM-1, MadCAM-1, CD51 (alpha V integrin), CD166 (ALCAM), CD324 (E-cadherin), CD49f (alpha6 integrin), CD104 (beta4 integrin), CD73, CD47, CD59, CD55 (DAF), CD81, CD9, CD29L, SSEA-4, CD109, CD298 (ATP1B3), and Na/K transporting ATPase subunit beta3. In some embodiments, the one or more cell surface markers is CD326 (EpCAM) or CD49f.

A surface molecule may be a human leukocyte antigen (HLA) protein. HLA is a class of cell surface antigens that encode the major histocompatibility complex (MHC), which regulates the human immune system. HLAs are generally classified into MHC class I and MHC class II. MHC class I, which includes HLA-A, HLA-B and HLA-C, present intracellular immunogenic peptides to immune cells. MHC class II present extracellular antigens to immune cells. In some embodiments, the hAECs express one or more HLA-Ia molecules. In some embodiments, the HLA-Ia molecule is HLA-A. In some embodiments, the hAECs express (a) HLA-G, (b) HLA-E, or (c) HLA-G and HLA-E. In some embodiments, the HLA-G is soluble HLA-G or the HLA-E is soluble HLA-E.

The disclosure is based, in part, on populations of cells that lack cells expressing HLA class II cell surface markers. In some embodiments, less than 2% (e.g., 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, or 0.1%) of the cells in the population express an HLA class II cell surface marker. In some embodiments, the hAECs do not express any HLA class II surface cell markers (e.g., surface antigens).

In some embodiments, a composition comprises at least 70%, at least 80%, at least 90%, at least 95%, at least 96%, at least 97%, at least 98%, or at least 99% hAECs, for example as measured according to the percentage of total cells in the composition. In some aspects, the disclosure provides a composition comprising a population of isolated cells comprising at least 97% human amniotic epithelial cells (hAECs); and a pharmaceutically acceptable excipient.

Aspects of the disclosure relate to populations of cells that have been cryopreserved. The disclosure is based, in part, on the recognition that compositions comprising populations of isolated amniotic cells that have been thawed after cryopreservation results, in some embodiments, in the population of cells being enriched for certain amniotic cell types (e.g.. hAECs) relative to other cell types in the population (e.g., hematopoietic cells, MSCs, endothelial cells, etc.). The viability of enriched cell populations (e.g. cell populations enriched in hAECs) may vary. In some embodiments, at least 80% of the hAECs in a population are viable after thawing. In some embodiments, at least 90%, 95%, or 99% of the hAECs in a population are viable after thawing.

In some embodiments, thawing comprises washing and resuspending a population of cells (e.g., a population of amniotic cells). Populations of cells may be resuspended in a buffer or pharmaceutically acceptable excipient (e.g., physiological saline) such that the resulting composition comprises a desired cell density. In some embodiments, the pharmaceutical composition comprises between about 1×10⁶ cells/mL and about 2×10⁷ cells/ml. In some embodiments, the pharmaceutical composition comprises at least 1×10⁷ hAECs/ml. In some embodiments, the pharmaceutical composition comprises a composition such as the isolated cell compositions described herein, including a cell composition that was cryopreserved and subsequently thawed.

In some embodiments, the pharmaceutically acceptable excipient comprises one or more of physiological saline, heparin, and human serum albumin (HSA). In some embodiments, the composition comprises less than 0.1% DMSO.

### Pharmaceutical Excipients

In some aspects, the disclosure provides a pharmaceutical composition made by a method described herein. In some embodiments, the composition comprises a population of enriched cells as described herein and a pharmaceutically acceptable excipient. As used herein the term “pharmaceutically acceptable excipient” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions can be prepared as described by Remington, J. P., & Allen, L. V. (2013). Remington: The science and practice of pharmacy. London: Pharmaceutical Press. The active ingredients may be admixed or compounded with any conventional, pharmaceutically acceptable carrier or excipient. The compositions may be sterile. In some embodiments, a pharmaceutically acceptable excipient is a saline solution. In some embodiments, a saline solution comprises between about 0.4% and about 1.0% w/v salt (e.g., NaCl). In some embodiments, the saline is physiological saline (e.g., 0.9% w/v NaCl, pH 5.4) supplemented with heparin (10 U/ml) and HSA (human serum albumin, 0.5%).

### Therapeutic Methods

In some aspects, the disclosure provides a method for treating a liver-associated disease, a vocal cord injury or a disease associated with a Complement Factor H deficiency in a subject in need thereof, the method comprising administering to the subject in need thereof a composition such as the isolated cell compositions described herein, including a cell composition that was cryopreserved and subsequently thawed.

In some embodiments, a subject is a mammal, for example a human, a mouse, a rat, a pig, a dog, a cat, a horse, a sheep, or a non-human primate. In some embodiments, the subject is not in a state of immunosuppression (e.g., currently being administered immunosuppressant drugs). In some embodiments, a subject is in a state of immunosuppression (e.g., the subject has been administered one or more immunosuppressive agents, or the subject has an immune disease or disorder resulting in immunosuppression).

Compositions described herein are useful, in some embodiments, for treating diseases associated with dysfunction of certain organs and/or tissues of a subject. In some embodiments, the tissues comprise liver, lung, pancreas, kidney, eye, trachea and vocal folds, esophagus, skin and hairs, cartilage, bone, brain and spinal cord, blood vessels, heart, uterus or immune system tissues.

In some embodiments, a subject has or is suspected of having a liver-associated disease. Generally, a liver-associated disease is a disease or disorder characterized by dysfunction of the liver. Examples of liver-associated diseases include but are not limited to acute liver disease (e.g., acute liver failure), chronic liver disease (e.g., chronic fibrosis of the liver), and certain inborn errors of metabolism, such as PKU). In some embodiments, the liver disease is acute/fulminant or chronic (e.g., fibrosis, cirrhosis, etc.). In some embodiments, the subject has or is suspected of having an inborn error of metabolism, such as PKU.

In some embodiments, a subject has or is suspected of having a vocal cord injury, such as vocal cord paralysis, vocal cord paresis, vocal cord infections, vocal cord tumors, vocal cord trauma (including surgical injury), vocal cord nodules, vocal cord polyps, contact ulcers and laryngitis.

In some embodiments, a subject has or is suspected of having a disease associated with a Complement Factor H deficiency. Examples of diseases associated with a Complement Factor H deficiency include, but are not limited to, membranoproliferative glomerulonephritis, atypical hemolytic uremic syndrome, macular degeneration, acute macular degeneration, neuropsychiatric disorders, schizophrenia, ischemic stroke, and complement-mediated renal conditions.

A subject that “has” a disease exhibits one or more signs or symptoms of the disease. For example, a subject having a liver-associated disease may be characterized by jaundice, abdominal pain or swelling, dark colored urine, chronic fatigue, nausea and vomiting, etc. A subject having PKU may be characterized by a musty odor, neurological problems (e.g., seizures), developmental delays, microcephaly, etc. A subject having a vocal cord injury may be characterized by hoarseness, low-pitched voice, breathy voice, loss of vocal range, throat pain, voice fatigue, change of vocal sound, croaking, loss of voice, breathing or swallowing difficulties, change of pitch, etc. A subject having a Complement Factor H deficiency may be characterized by renal failure, recurrent bacterial infections, hematuria, thickening of the glomerular basement membrane on renal biopsy, hypocomplementemia, decreased serum Complement Factor H, etc. A subject “suspected of having” a disease may or may not exhibit one or more signs of symptoms of the disease. In some embodiments, a subject suspected of having a disease comprises one or more genetic mutations (e.g., substitutions, insertions, deletions, etc.) that are associated with a disease and/or associated with aberrant function of the gene product encoded by the gene. In some embodiments, a subject suspected of having PKU comprises one or more mutations in a phenylalanine hydroxylase gene (PAH), or is characterized by having reduced function or activity of PAH protein.

As used herein, the term “treating” refers to the application or administration of a composition (e.g., a composition as described herein), to a subject who has a disease or disorder associated with the liver (e.g., acute liver failure, fibrosis, PKU, etc.), with a vocal cord injury (e.g. vocal cord infection, laryngitis, etc.), or with a Complement Factor H deficiency (e.g. membranoproliferative glomerulonephritis, atypical hemolytic uremic syndrome, etc.), with the purpose to cure, heal, alleviate, relieve, alter, remedy, ameliorate, improve, or affect the disorder, the symptom of the disease, or the predisposition toward the disease.

Alleviating a disease or disorder associated with the liver (e.g., acute liver failure, fibrosis, PKU, etc.), with a vocal cord injury (e.g., vocal cord infection, laryngitis, etc.), or with a Complement Factor H deficiency (e.g. membranoproliferative glomerulonephritis, atypical hemolytic uremic syndrome, etc.) includes delaying the development or progression of the disease, or reducing disease severity. Alleviating the disease does not necessarily require curative results. As used therein, “delaying” the development of a disease (such as disease or disorder associated with the liver (e.g., acute liver failure, fibrosis, PKU, etc.), with a vocal cord injury (e.g. vocal cord infection, laryngitis, etc.), or with a Complement Factor H deficiency (e.g. membranoproliferative glomerulonephritis, atypical hemolytic uremic syndrome, etc.)) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset of a disease or disorder associated with the liver (e.g., acute liver failure, fibrosis, PKU, etc.), with a vocal cord injury (e.g. vocal cord infection, laryngitis, etc.), or with a Complement Factor H deficiency (e.g. membranoproliferative glomerulonephritis, atypical hemolytic uremic syndrome, etc.).

The compositions are administered in sufficient amounts to transfer the cells to a desired tissue (e.g., liver tissue, vocal cord) and to provide sufficient levels of engraftment, differentiation, and expansion. In some embodiments, administration is performed via systemic injection, for example intravenous injection, intra-arterial injection, intraperitoneal injection, intravascular injection, intracerebral administration, intramuscular injection, etc. In some embodiments, administration is via direct injection to a target tissue, for example liver tissue, lung tissue, vocal cord, etc. In some embodiments, administration is performed via topical administration. In some embodiments, the administration is via direct injection to the liver of the subject. In some embodiments, the direct injection is portal vein injection, hepatic artery injection, or mesenteric vein injection. In some embodiments, the administration results in engraftment of about 2% to about 100% hAECs in the composition in the liver tissue of the subject. In some embodiments, the administration results in engraftment of about 4% to about 10% of the hAECs in the composition in the target tissue (e.g. liver tissue, vocal cord, etc.) of the subject. In some embodiments, the administration results in engraftment of about 4%, 5%, 6%, 7%, 8%, 9% or 10% of the hAECs in the composition in the target tissue (e.g. liver tissue, vocal cord, etc.) of the subject. In some embodiments, the administration results in engraftment of about 5% to about 20% of the hAECs in the composition in the target tissue (e.g. liver tissue, vocal cord, etc.) of the subject. In some embodiments, the administration results in engraftment of about 5%, 6%, 7%, 8%, 9%, 10%, 11%, 12%, 13%, 14%, 15%, 16%, 17%, 18%, 19% or 20% of the hAECs in the composition in the target tissue (e.g. liver tissue, vocal cord, etc.) of the subject.

Aspects of the disclosure relate to methods of treating an inborn error of metabolism in a subject. In some embodiments, administration of compositions described herein results in hAEC engraftment in the subject, and subsequent differentiation into hepatocytes in the subject's liver. In some embodiments, the differentiated hepatocytes express one or more gene products (e.g., proteins, such as enzymes) that are not expressed by the endogenous hepatocytes of the subject. In some embodiments, the gene product is an enzyme of interest (e.g., an enzyme in which the subject is deficient), for example PAH. In some embodiments, the administration results in an increase (relative to the subject prior to the administration) of expression of the enzyme of interest (e.g., PAH) in the liver or blood of the subject. In some embodiments, the administration results in an increase (relative to the subject prior to the administration) of expression of the enzyme of interest (e.g., PAH) expression in the brain of the subject. Levels of enzyme (e.g., PAH) in a sample obtained from the subject, for example a blood sample or CSF sample, may be measured by any methods known in the art. In some embodiments, the administration results in a reduction of the level of a substrate of the enzyme of interest (e.g., PHE in the case of PAH) in the blood of the subject relative to the level in the subject's blood prior to the administration. In some embodiments, the substrate (e.g., PHE) level is reduced by between about 10% and about 50% in the blood of the subject. In some embodiments, the substrate (e.g., PHE) level is reduced by between about 75% and about 95% in the brain of the subject.

### EXAMPLE

### Example 1

This example describes one embodiment of a protocol for extraction, cryopreservation, and reconstitution of human amniotic epithelial cells (hAECs). The embodiment is described as follows.

### Surgical Dissection

Wearing sterile gloves, transfer a whole placenta in the sterile tray. To flat the placenta leaves down, use sterile scissors to cut it in 3-4 region, and flatten it down like flower petals. Trim the umbilical cord close to the amnion surface and perform 3 superficial surgical incisions into, but not through, the fetal surface, to delimitate 6 slices on the placenta. The incisions do not include the distal part of the amnion membrane. Prepare 3 liters of Ringers solution supplemented with 5 mM EGTA. Pour one liter of Ringers solution supplemented with EGTA on top of the membrane and gently remove blood clots. Using sterile forceps and/or pinching the amnion membrane between the fingers peel the amnion membrane from the underlying chorion layer. Start from the cut edge, in the region of the umbilical cord (middle of the placenta body) and peel the membrane from the placenta. Place the pieces of amnion (6 or more) in a pre-weighted sterile plastic container containing Saline solution, seal with lid, and record the weight. The difference in weight represents the amount of wet amnion tissue (typically ranging from about 8 g to 15 g). Discard the remaining part of the placenta, aspirate blood and Ringers solution from metal tray and remove the dissecting material from the work area (metal tray and scalpel). Pour ˜300 ml of Ringers-EGTA in 3 sterile 500-ml glass beakers and ˜200 ml in a sterile metal basin. Transfer the amnion in the sterilized 500-ml glass beaker containing Ringers-EGTA solution and using forceps wash it from the blood. If Ringers-EGTA solution turns red, transfer the tissue in a second beaker and keep washing it. Transfer tissue in the sterile metal basin and by flatting it down identify regions with blood clots and carefully removed all of them using forceps and scissors. No obvious blood/blood clots should remain. Cut the amnion membrane in approximately 1.5-2 gr of tissue (2-3 ml volume), and transfer into a Saline-containing beaker. Repeat washing steps for every amnion tissue. At the end of the procedure there should be >6 pieces of clean tissue immerse in the saline. If Ringers-EGTA solution is turning into a vivid red color, discard and replace with fresh solution.

### Enzymatic Digestion

Place the amnion tissues in 50-ml polypropylene tubes (approx. 1.5-2 gr of wet tissue per tube). Add 25 ml of TrypLE 10× solution per tube (total number of tubes=6-10). Incubate 30 min at 37° C. in a rotor (IncubatorGenie; Scientific Industries, Inc.) at 35 rpm speed. Shake vigorously every tube after 10 and 20 minutes of digestion. After 30 minutes, shake vigorously every tube and pour membrane and enzyme solutions in a sterile 500-ml beaker containing ˜200 ml Saline solution, and wash the tissue using sterile forceps to agitate the tissue to release cells. Re-distribute the membrane into the 50-ml tubes, add ˜30 ml Saline solution and vigorously shake the tubes. Pour the content in a second sterile 500-ml beaker containing ˜100 ml Saline solution. Discard the membrane. In new tubes, combine all the solutions, add human albumin to a final concentration equal to 0.5%-1% in every tube and centrifuge for 5 min at 1000× g, 4° C. Remove the supernatant, resuspend and combine the pellets in 80-100 ml Saline solution. Divide the cell suspension in two 50 ml tubes and centrifuge for 5 min at 300× g, 4° C. Swirl the tubes in order to release the cells, do not shake. Resuspend and combine the pellets into 40-45 ml of Saline solution and filter the cell preparation through a 100-μm filter. Count cell number and viability using the Trypan Blue exclusion (TBE) method with a Burker chamber.

### Cryopreservation

The final cell suspension from above is centrifuged (300× g, 4° C.) and gently resuspended at a density of 10⁷/ml in DMSO-free cryogenic solution (e.g., PRIME-XV MSC FreezIS DMSO-Free (Irvine Scientific/Fujifilm), etc.), and transferred to pre-labelled cryogenic vials or cryogenic bags. Vials are quickly moved to a controlled-rate, alcohol-free cell freezing container (CoolCell or similar), which provides a freezing rate of −1° C./minute when transferred to a −80° C. freezer. After 90 minutes the vials are transferred to a −150° C. freezer or a liquid nitrogen container for long-term storage.

### Example 2

### Materials and Methods

### hAEC Isolation Procedure

The human placenta was procured from uncomplicated full-term pregnancy from healthy mothers. For sterility purposes, placenta derived following cesarean section were used in this study, but similar procedure can be applied to natural delivery placenta. A protocol for hAEC isolation was performed (e.g., as described above).

### Cryopreservation Procedure

hAEC were not maintained in culture or selected ex vivo prior to freezing. Following cell isolation, hAEC were immediately re-suspended in cryogenic solution (University of Wisconsin solution supplemented with 10% DMSO), at a cell density of 10⁷ viable cells/mL. Every cell batch was aliquoted into 1.5 mL Cryotubes (Corning, NY, USA) and placed in a controlled freezing container that lowered the temperature by 1° C. per minute when stationed in −80° C. freezer (Biocision, Larkspur, Calif., USA). The cells were stored in the vapor phase of a liquid nitrogen storage tank. The cells were kept in liquid nitrogen a minimum of 30 days and a maximum of 36 months before analysis.

### Thawing Procedure

The cryovials were removed from the liquid nitrogen tank and rapidly thawed by partial emersion in a water bath maintained at 37° C., until small ice crystals remain (commonly for 80-120 seconds). Cells were diluted into 10 volumes of ice cold Plasmalyte solution (Baxter, Norfolk, UK) (or similar), supplemented with 2% human serum albumin, and centrifuged at 300× g for 5 minutes. Cell pellet was resuspended in cold Plasmalyte solution (or similar) and filtered through a 100 μm cell strainer. Cell viability and recovery were determined by TBE.

### Flow Cytometry Analysis

The heterogeneity of the cell suspension is evaluated based on surface markers quantified by flow cytometric analysis (FACS) or similar cytostaining techniques. Both freshly isolated and cryopreserved hAEC are incubated with monoclonal antibodies directed against cell-specific surface protein, properly diluted in PBS solution and incubated for 30 min at 4° C. The human-specific antibodies included in the study were: CD326 (Clone-HEA-125; Miltenyi Biotech); CD31 (Clone WM59), CD44 (Clone G44-26), CD45 (Clone-T29/33), CD49f (Clone-GoH3), CD105 (Clone-SN6; all from BD Biosciences, San Jose, Calif., USA). All the 6 monoclonal antibodies were directly conjugated with one of three specific dyes, Fluorescein isothiocyanate (FITC) or Phycoerythrin (PE) or Allophycocyanin (APC) to perform multilineage evaluation on the same suspension. Corresponding isotype controls were also analyzed. The cells were washed and fixed with 2% BD™ stabilizing fixative (BD Biosciences) for 10 min at room temperature. Cells were washed and re-suspended in ice cold PBS, and analyzed on a FACSCanto (BD Biosciences) using FlowJo™_V10 software.

### Gene Profiling by qPCR

Thawed hAEC were lysed in Trizol™ solution (Life Tech, Carlsbad, Calif., USA) and total RNA was isolated according to the manufacturer's instructions. Total RNA was converted to complimentary DNA using high-capacity cDNA kit (Life tech, Carlsbad, Calif., USA). Gene expression was assessed using TaqMan assays for DLK-1 (HS00171584), MMP2 (HS1548728), MMP3 (HS00968305), MMPI (HS1042812), MMP8 (HS01029057), MMP9 (HS00957562), MMP 12 (HS00159181), MMP13 (HS00942591), TIMP1 (HS01092512), TERT (HS00972650), OCT4 (HS04260367), NANOG (HS04260366), SOX2 (HS01053049), CD73 (HS00159686), CD39 (HS00969559), CD38 (HS01120071), IDO (HS00984148), HLA-G (HS00365950), HLA-E (HS03045171), HLA-F (HS04185703). Reactions were run in duplicate with human cyclophilin A (PPIA) (Hs99999904_m1) as a house keeping gene as control for all experiments. Calculation of relative levels of expression were done according to the comparative Ct-method as follows: 2(−ΔCt), where ΔCt=(Ct gene of interest−Ct internal control). Ct values for the gene of interest 35 or higher were considered as unreliable and ignored from the calculation.

### Viability of Thawed Cells

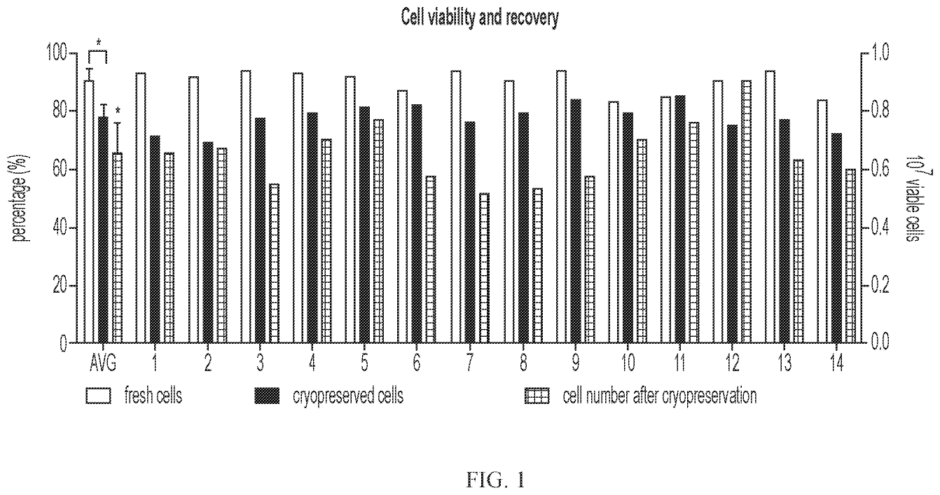

Cell viability measured immediately after isolation was 90±4% (n=14). When cells from the same 14 cases were thawed months to years later, the average cell viability was significantly lower (78%±5%; p<0.0001; FIG. 1). Ten million viable hAECs were initially cryopreserved. On average, when different cases of hAEC were thawed, a significantly lower number of cells were recovered (6.5±1.1 million/ml; p<0.00001), corresponding to 55-95% of the initially cryopreserved cells. Cases characterized with the highest viability post-cryogenic procedure did not always result in highest cell recovery (FIG. 1).

### Quality Control of Cryopreserved Cell Products

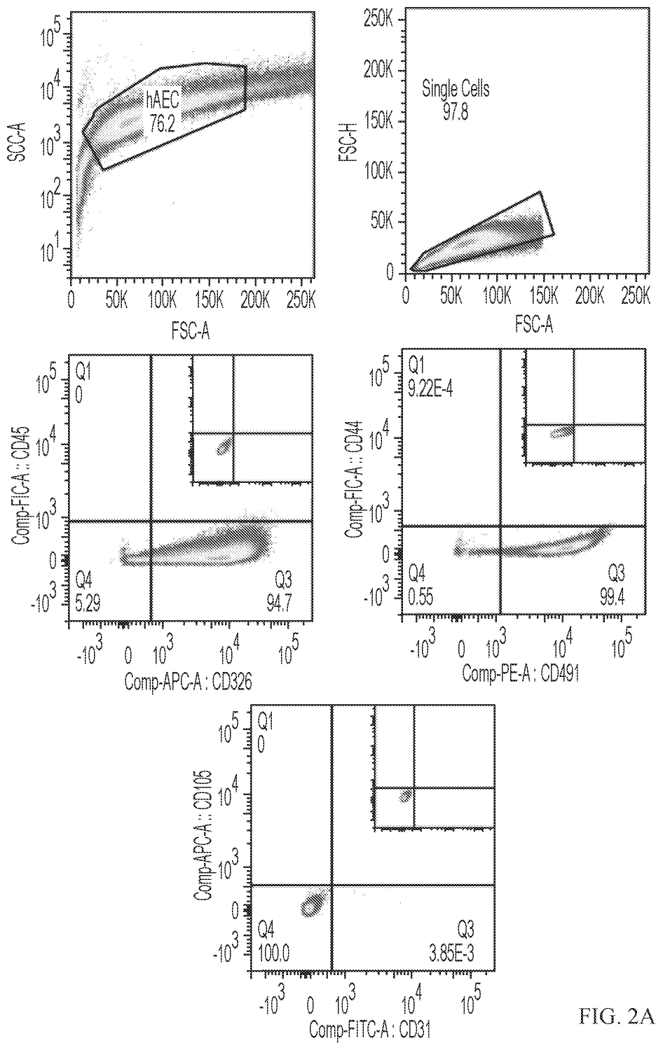

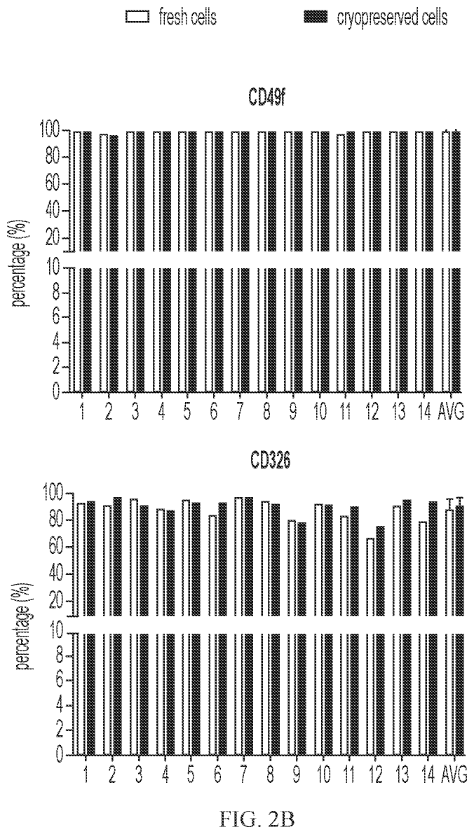

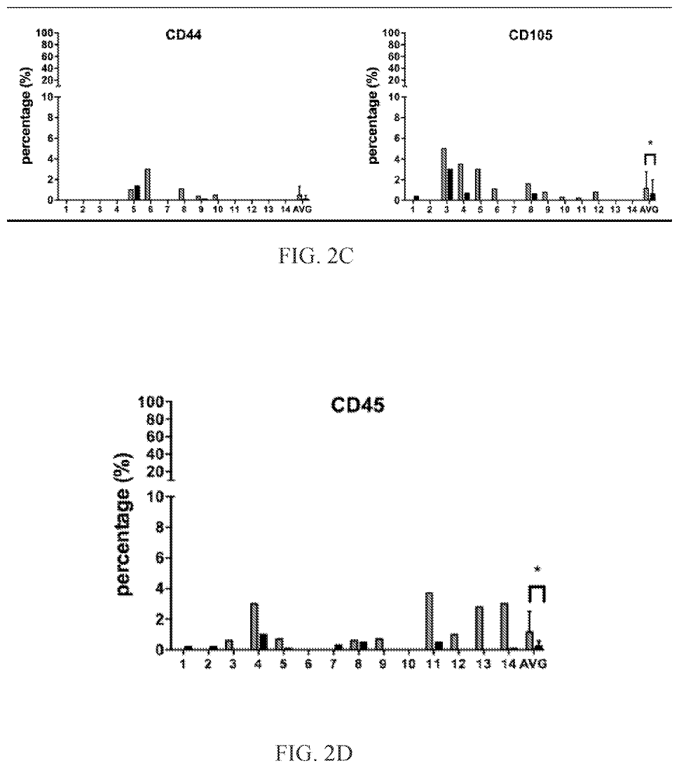

There is some heterogeneity in the types of cells recovered from amnion membrane depending on the isolation procedure and the reagents used. Preparations of hAEC are generally characterized for the presence of static surface markers to identity different cell types in amnion-derived cell product. High-speed fluorescence-activated cell sorting (FACS) was performed on each hAEC sample, before and after cryopreservation (FIG. 2A). Specific epithelial cell markers (CD49f and CD326) were present on the majority of isolated cells. (FIG. 2B). The average expression of CD49f (alpha 6 integrin subunit) on hAEC was unaffected by cryopreservation procedure and it was expressed on 99±1% of freshly isolated as well as cryopreserved cells (p=0.1386) (FIG. 2B). Similarly, the expression of another epithelial marker, CD326 (EpCAM), was different between fresh (88%±8%) and cryopreserved cells (91±6%; p=0.0939) (FIG. 2B).

Digestion of amnion membrane to release epithelial cells results, in some instances, in stromal cell release from inner layer of the amnion membrane. The presence of amnion-derived MSC was investigated with common stromal markers, CD105 and CD44. Approximately 50% of the fresh (e.g. non-cryopreserved) hAEC preparations contained cells with MSC surface markers. Cells positive for CD44 were found on 3/14 preparations, on an average of 0.5±0.8% of the total cell number (FIG. 2C), while CD105 (endoglin) was expressed in 9/14 preparations on 1.2±1.6% of the cells. After cryopreservation, stromal marker positive cells were below the limits of detection on 9/14 hAEC (FIG. 2C): with 0.1±0.4% (p=0.173) and 0.6±1.4% (p=0.0128) of the total cells remaining positive for CD44 and CD105, respectively.

Hematopoietic or endothelial cells may be present in the final cell suspension from residual contamination with blood or digestion of placentae vascular tissue. The presence of CD45, a receptor linked to protein tyrosine phosphatase present in cells of the hematopoietic lineage was detected in 9/12 of hAEC preparations immediately after isolation at an average level of 1.2±1.4% cells, but was greatly reduced after cryopreservation and washing steps, where only 4 preparations still contained CD45 positive cells (0.3%±0.4%; p=0.0146) (FIG. 2D). The endothelial cell marker (CD31) was undetectable in all 14 samples, after isolation or cryopreservation.

### Immunomodulatory Molecules

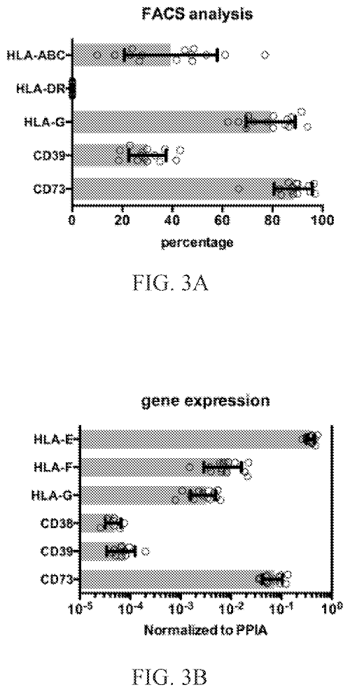

Mismatches in human leukocyte antigens (HLA) expression are recognized by immune cells and generally induce rejection. The expression of classical HLA-Ia antigens (HLA-A,-B,-C) and the HLA-II were quantified (FIG. 3A). Expression of HLA-Ia was detected on an average of 39±18% of the cells, with a variable range of expression in different cryopreserved hAEC cases (10-55%). As previously reported, human leukocyte antigens class II was negative on all hAEC, before and after cryopreservation.

Amnion epithelial cells have been observed to express characteristic immunomodulatory molecules, such as HLA class Ib. These and other surface proteins have been ascribed as mediators in immune-recognition and reactions against innate or adaptive immune cells. The presence of HLA-G on hAEC after isolation was confirmed using a specific antibody, and HLA-G expression was maintained after cryopreservation on 79±9% of the cells (range, 66-91%) (FIG. 3A). HLA-Ib expression by hAEC was investigated by transcriptome analysis: HLA-G, -E and -F forms were detected in all the hAEC samples analyzed, with HLA-E expression approximately 100-fold higher than HLA-G and HLA-F (FIG. 3B). Certain ectoenzymes, such as adenosinergic enzymes CD39 and CD73, play an activate role in modulation of the immune system. Both HLA molecules and ectoenzymes CD39 and CD73 (30±7% and 88±7%, respectively) were expressed on all 14 samples of hAEC after thawing (FIG. 3A). As with HLA-Ib, hAEC still expressed ectonucleotidase enzymes CD38, CD39 and CD73 post cryopreservation (FIG. 3B). Indoleamine-pyrrole 2,3-dioxygenase (IDO), another enzyme involved in immune modulation and immune tolerance by limiting T-cell function and described in immunomodulatory cells, was also analyzed but was undetectable in all hAEC samples.

### Pluripotency Genes

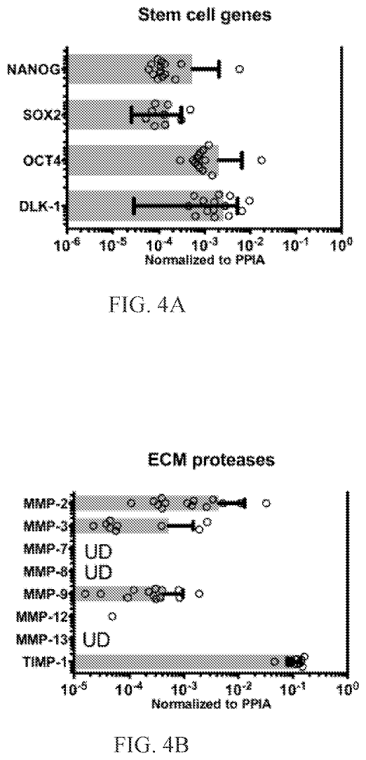

Multipotency is another important characteristic ascribed to hAECs. The expression of the transcription factors OCT-4, NANOG, and SOX2 are critical in the maintenance of pluripotency, and all three transcription factors were expressed in all hAEC samples (FIG. 4A). Another transmembrane protein evolutionarily conserved and responsible for several developmental processes such as cellular fate determination and terminal differentiation, delta-like 1 homolog (DLK-1), was found to be expressed in all cryopreserved preparations (FIG. 4A). Telomerase reverse transcriptase (TERT), a component of telomerase complex responsible for long-term survival and repetitive cycles of cell replication, was undetectable in all hAEC cases (data not shown).

### Cell Engraftment Enzymes

Transplanted epithelial cells typically require integration into parenchyma to establish cell-to-cell and cell-to-ECM interaction supporting their survival and growth. Integration and nidation is in part facilitated by the secretion of matrix metalloproteinases (MMP), enzymes that degrade ECM and enhance cell adhesion, migration, and proliferation. Several MMPs were analyzed in the hAEC samples. MMP2, MMP3 and MMP9 were expressed in all cryopreserved samples (FIG. 4B). Additional isoforms, MMP7, MMP8 and MMP13, were also analyzed but were undetectable in all hAEC samples (FIG. 4B). Low level expression of MMP12 was detected in one sample, however, it was undetectable in the other 13 cases. Finally, the tissue inhibitor of metalloproteinases TIMP1 was highly expressed in all cryopreserved preparations (FIG. 4B).

### Example 3

### Materials and Methods

### hAEC Isolation Procedure

The human placenta was procured from uncomplicated full-term pregnancy from healthy mothers. For sterility purposes, placenta derived following cesarean section were used in this study, but similar procedure can be applied to natural delivery placenta. A protocol for hAEC isolation was performed as described in Example 1, wherein TrypLE used for the digestion step was replaced with a protease mixture supplemented with EGTA. In some embodiments, supplementing the protease mixture with EGTA significantly improved the number of hAECs released from the amniotic membrane into the solution.

What is claimed is:

1. A composition comprising:
(a) a population of isolated cells comprising at least 70% human amniotic epithelial cells (hAECs), wherein no more than 2% of the cell population is hematopoietic cells, and less than 1% of the remaining cells are negative for HLA-DR; and
(b) a pharmaceutically acceptable excipient.

2. The composition of claim 1, wherein the isolated cells are capable of adhering to an extracellular matrix (ECM) protein, optionally wherein the ECM protein is a laminin or collagen isoform, fibronectin, elastin, gelatin, hyaluronic acid or hyaluronic acid-binding protein, and/or Engelbreth-Holm-Swarm (EHS) Matrix Extract.

3. The composition of claim 1, wherein less than 30% of the cells in the population express a stromal cell marker.

4. The composition of claim 3, wherein the stromal cell marker is CD44 or CD105.

5. The composition of any one of claims 1 to 4, wherein the hAECs express one or more cell surface markers selected from CD326 (EpCAM), CD29 (betal-integrin), CD106 (VCAM-1), MadCAM-1, CD51 (alphaV integrin), CD166 (ALCAM), CD324 (E-cadherin), CD49f (alpha6 integrin), CD104 (beta4 integrin), CD73, CD47, CD59 (DAB), CD55 (DAF), CD81, CD9, CD29L, SSEA-4, CD109, CD298 (ATP1B3), and Na/K transporting ATPase subunit beta3.

6. The composition of claim 5, wherein at least one of the cell surface markers is CD326 or CD49f.

7. The composition of any one of claims 1 to 6, wherein the hAECs express one or more HLA-Ia molecules.

8. The composition of claim 7, wherein the HLA-Ia molecule is HLA-A.

9. The composition of any one of claims 1 to 8, wherein the hAECs express (a) HLA-G, (b) HLA-E, or (c) HLA-G and HLA-E.

10. The composition of claim 9, wherein the HLA-G is soluble HLA-G or the HLA-E is soluble HLA-E.

11. The composition of any one of claims 1 to 10, wherein the hAECs do not express any HLA class II antigens.

12. The composition of any one of claims 1 to 11, wherein the population of cells has previously been cryopreserved and subsequently thawed.

13. The composition of claim 12, wherein at least 80% of the hAECs are viable after thawing.

14. The composition of claim 13, wherein at least 90%, 95%, or 99% of the hAECs are viable after thawing.

15. The composition of any one of claims 1 to 14, wherein the hAECs are present at a density between about 1×10⁶ cells/ml and about 1×10⁸ cells/ml.

16. The composition of any one of claims 1 to 15, wherein the pharmaceutically acceptable excipient comprises at least one of the following: physiological saline, heparin, and human serum albumin (HSA).

17. The composition of any one of claims 1 to 16, wherein the composition comprises less than 0.1% DMSO.

18. A method for producing a cell preparation comprising an enriched population of human amniotic epithelial cells (hAECs), the method comprising:
(a) combining amniotic membrane tissue with an enzyme under shaking conditions that result in tissue digestion and release of a population of cells;
(b) suspending the cells in a first buffer, optionally wherein the first buffer comprises albumin;
(c) removing the first buffer (and, optionally, albumin) and resuspending the population of cells in a saline solution, thereby producing a cell preparation comprising hAECs;
(d) filtering the cell preparation produced into one or more containers comprising a DMSO-free cryogenic solution to produce a cell preparation comprising an enriched population of human amniotic epithelial cells (hAECs); and, optionally,
(e) placing the one or more containers in a temperature-controlled storage apparatus, wherein the temperature of the apparatus is reduced at a rate of 1° C. per minute until a final temperature of lower than −80° C. is reached.

19. The method of claim 18, wherein the final temperature is maintained for a minimum of 30 days.

20. The method of claim 18 or 19, wherein the digesting comprises incubating the amniotic membrane tissue with trypsin.

21. The method of claim 18 or 19, wherein the digesting comprises incubating the amniotic membrane tissue with a protease, or a protease mixture.

22. The method of claim 18 or 19, wherein the digesting comprises incubating the amniotic membrane tissue with a protease that cleaves integrin.

23. The method of claim 21 or 22, wherein the protease or protease mixture further comprises a chelating agent, optionally wherein the chelating agent comprises or consists of EGTA or EDTA.

24. The method of any one of claims 18 to 23, further comprising thawing the cell preparation to between 4° C. and room temperature.

25. The method of claim 24, further comprising removing the cryoprotectant and resuspending the cell preparation in a pharmaceutically acceptable excipient to produce a pharmaceutical composition.

26. The method of claim 25, wherein the pharmaceutical composition comprises at least 70% human amniotic epithelial cells (hAECs), wherein no more than 2% of the cell population is hematopoietic cells, and less than 1% of the remaining cells are negative for HLA-DR.

27. The method of claim 25 or 26, wherein the pharmaceutical composition comprises at least about 5×10⁶ hAECs.

28. A pharmaceutical composition made by the method of any one of claims 18 to 27.

29. A method for treating a liver-associated disease, a vocal cord injury or a disease associated with a Complement Factor H deficiency in a subject in need thereof, the method comprising administering to the subject in need thereof the composition of any one of claims 1 to 17, or the pharmaceutical composition of claim 28.

30. The method of claim 29, wherein the liver disease is acute liver disease or chronic liver disease.

31. The method of claim 29 or 30, wherein the subject is a mammal.

32. The method of any one of claims 29 to 31, wherein the subject is a human.

33. The method of any one of claims 29 to 32, wherein the administration is via direct injection to the liver of the subject.

34. The method of claim 33, wherein the direct injection is portal vein injection, hepatic artery injection or mesenteric vein injection.

35. A method for treating phenylketonuria (PKU) in a subject in need thereof, the method comprising administering to the subject having PKU the composition of any one of claims 1 to 17, or the pharmaceutical composition of claim 28.

36. The method of claim 35, wherein the administration results in expression of phenylalanine hydroxylase (PAH) enzyme in the liver of the subject.

37. The method of claim 35 or 36, wherein the administration results in a reduction of phenylalanine (PHE) level in the blood of the subject relative to the PHE level in the subject's blood prior to the administration.

38. The method of any one of claims 35 to 37, wherein the subject is not in a state of immunosuppression prior to the administration.

39. The method of any one of claims 35 to 38, wherein the subject is a human.

40. The method of any one of claims 35 to 39, wherein the administration is via direct injection to the liver of the subject.

41. The method of claim 40, wherein the direct injection is portal vein injection or mesenteric vein injection.

42. A composition comprising:
(a) a population of isolated cells comprising at least 90% human amniotic epithelial cells (hAECs); and
(b) a pharmaceutically acceptable excipient.

43. The composition of claim 42, wherein less than 2% of the cells in the population express a stromal cell marker.

44. The composition of claim 43, wherein the stromal cell marker is CD44 or CD105.

45. The composition of any one of claims 42 to 44, wherein less than 1% of the cells in the population express a hematopoietic cell marker.

46. The composition of claim 44, wherein the hematopoietic cell marker is CD45.

47. The composition of any one of claims 42 to 46, wherein the hAECs express one or more cell surface markers selected from CD326 (EpCAM), CD29 (betal-integrin), CD106 (VCAM-1), MadCAM-1, CD51 (alphaV integrin), CD166 (ALCAM), CD324 (E-cadherin), CD49f (alpha6 integrin), CD104 (beta4 integrin), CD73, CD47, CD59 (DAB), CD55 (DAF), CD81, CD9, CD29L, SSEA-4, CD109, CD298 (ATP1B3), and Na/K transporting ATPase subunit beta3.

48. The composition of claim 47, wherein at least one of the epithelial cell markers is CD326 or CD49f.

49. The composition of any one of claims 42 to 48, wherein the hAECs express one or more HLA-Ia molecules.

50. The composition of claim 49, wherein the HLA-Ia molecule is HLA-A.

51. The composition of any one of claims 42 to 50, wherein the hAECs express (a) HLA-G, (b) HLA-E, or (c) HLA-G and HLA-E.

52. The composition of claim 51, wherein the HLA-G is soluble HLA-G or the HLA-E is soluble HLA-E.

53. The composition of any one of claims 42 to 52, wherein the hAECs do not express any HLA class II antigens.

54. The composition of any one of claims 42 to 53, wherein the population of cells has previously been cryopreserved and subsequently thawed.

55. The composition of claim 54, wherein at least 80% of the hAECs are viable after thawing.

56. The composition of claim 55, wherein at least 90%, 95%, or 99% of the hAECs are viable after thawing.

57. The composition of any one of claims 42 to 56, wherein the hAECs are present at a density between about 1×10⁶ cells/ml and about 1×10⁸ cells/ml.

58. The composition of any one of claims 42 to 57, wherein the pharmaceutically acceptable excipient comprises at least one of the following: physiological saline, heparin, and human serum albumin (HSA).

59. The composition of any one of claims 42 to 58, wherein the composition comprises less than 0.1% DMSO.

60. A method for producing a cell preparation comprising an enriched population of human amniotic epithelial cells (hAECs), the method comprising:
(a) combining amniotic membrane tissue with an enzyme under shaking conditions that result in tissue digestion and release of a population of cells;
(b) suspending the cells in a first buffer, optionally wherein the first buffer comprises albumin;
(c) removing the first buffer (and, optionally, albumin) and resuspending the population of cells in a saline solution, thereby producing a cell preparation comprising hAECs;
(d) filtering the cell preparation produced into one or more containers comprising a DMSO-free cryogenic solution to produce a cell preparation comprising an enriched population of human amniotic epithelial cells (hAECs); and, optionally,
(e) placing the one or more containers in a temperature-controlled storage apparatus, wherein the temperature of the apparatus is reduced at a rate of 1° C. per minute until a final temperature of lower than −80° C. is reached.

61. The method of claim 60, wherein the final temperature is maintained for a minimum of 30 days.

62. The method of claim 60 or 61, wherein the digesting comprises incubating the amniotic membrane tissue with trypsin.

63. The method of claim 60 or 61, wherein the digesting comprises incubating the amniotic membrane tissue with a protease, or any protease mixture.

64. The method of claim 60 or 61, wherein the digesting comprises incubating the amniotic membrane tissue with a protease that cleaves integrin.

65. The method of claim 63 or 64, wherein the protease or protease mixture further comprises a chelating agent, optionally wherein the chelating agent comprises or consists of EGTA or EDTA.

66. The method of any one of claims 60 to 65, further comprising thawing the cell preparation to between 4° C. and room temperature.

67. The method of claim 66, further comprising removing the cryoprotectant and resuspending the cell preparation in a pharmaceutically acceptable excipient to produce a pharmaceutical composition.

68. The method of claim 67, wherein the pharmaceutical composition comprises at least 90% human amniotic epithelial cells (hAECs).

69. The method of claim 67 or 68, wherein the pharmaceutical composition comprises at least about 5×10⁶ hAECs.

70. A pharmaceutical composition made by the method of any one of claims 60 to 69.

71. A method for treating a liver-associated disease, a vocal cord injury or a disease associated with a Complement Factor H deficiency in a subject in need thereof, the method comprising administering to the subject in need thereof the composition of any one of claims 42 to 59, or the pharmaceutical composition of claim 70.

72. The method of claim 71, wherein the liver disease is acute liver disease or chronic liver disease

73. The method of claim 71 or 72, wherein the subject is a mammal.

74. The method of any one of claims 71 to 73, wherein the subject is a human.

75. The method of any one of claims 71 to 74, wherein the administration is via direct injection to the liver of the subject.

76. The method of claim 75, wherein the direct injection is portal vein injection, hepatic artery injection or mesenteric vein injection.

77. A method for treating phenylketonuria (PKU) in a subject in need thereof, the method comprising administering to the subject having PKU the composition of any one of claims 42 to 59, or the pharmaceutical composition of claim 70.

78. The method of claim 77, wherein the administration results in expression of phenylalanine hydroxylase (PAH) enzyme in the liver of the subject.

79. The method of claim 77 or 78, wherein the administration results in a reduction of phenylalanine (PHE) level in the blood of the subject relative to the PHE level in the subject's blood prior to the administration.

80. The method of any one of claims 77 to 79, wherein the subject is not in a state of immunosuppression prior to the administration.

81. The method of any one of claims 77 to 80, wherein the subject is a human.

82. The method of any one of claims 77 to 81, wherein the administration is via direct injection to the liver of the subject.

83. The method of claim 82, wherein the direct injection is portal vein injection or mesenteric vein injection.
